(19) United States
(12) Patent Application Publication
(10) Pub. No.: US 2014/0128771 A1
(43) Pub. Date: May 8, 2014

(54) VITRECTOMY AND BIOPSY SYSTEM AND METHODS OF ASSEMBLING SAME

(71) Applicant: Synergetics, Inc., O'Fallon, MO (US)

(72) Inventors: Matthew Paul LaConte, Maryland Heights, MO (US); Carl C. Awh, Nashville, TN (US); James C. Easley, Cottleville, MO (US); Matthew A. Hanlon, O'Fallon, MO (US)

(21) Appl. No.: 14/075,856

(22) Filed: Nov. 8, 2013

Related U.S. Application Data
(60) Provisional application No. 61/724,084, filed on Nov. 8, 2012.

(51) Int. Cl. A61B 10/02; A61F 9/007

(52) U.S. Cl. CPC A61B 10/0266; A61F 9/00736
USPC 600/566; 29/428

(57) ABSTRACT

A biopsy system includes a cutting probe including a suction port, a suction line connected to the suction port, and a biopsy chamber fluidly connected to the cutting probe by the suction line. The biopsy chamber is positioned proximate the cutting probe such that the biopsy chamber is configured to receive and store a biological sample free from irrigation fluid.

### CROSS-REFERENCE TO RELATED APPLICATIONS

This application claims the benefit of U.S. Provisional Application No. 61/724,084, filed Nov. 8, 2012, the entirety of which is hereby incorporated by reference.

### FIELD

This disclosure generally relates to surgical devices and, more specifically, to a vitrectromy and biopsy system for removing samples from the body during surgery.

### BACKGROUND

Conventional surgical instruments may be used to remove or “biopsy” a portion of the body. In the case of ophthalmic procedures, all or part of the vitreous in the eye may be removed, e.g., for testing or treatment. The vitreous is a colorless transparent jelly-like material that fills the area of the eye posterior to the crystalline lens. The vitreous is filled with numerous fibers that are often attached to the retina. Removal of the vitreous is difficult due to the presence of the fibers and the possibility of detachment of the inflexible and very delicate retina.

The conventional instruments require hand or manually operated suction to remove vitreous from the eye, e.g., with a syringe. Further, conventional instruments typically require the eye to be irrigated during this suction to prevent collapse during vitreous removal, and this irrigation dilutes the vitreous sample. Accordingly, a better system to remove undiluted vitreous from the eye is needed.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

In one aspect, a biopsy system includes a cutting probe including a suction port, a suction line connected to the suction port, and a biopsy chamber fluidly connected to the cutting probe by the suction line. The biopsy chamber is positioned proximate the cutting probe such that the biopsy chamber is configured to receive and store a biological sample. In some embodiments, the biopsy chamber is configured to receive a vitreous sample free from irrigation fluid.

In another aspect, a method of assembling a biopsy system is disclosed. The biopsy system includes a cutting probe having a suction port, a suction line having a first section and a second section, and a biopsy chamber having a first end and a second end. The method includes fluidly connecting the first end of the biopsy chamber to the suction port of the cutting probe with the first section of the suction line, connecting the second end of the biopsy probe to the second section of the suction line, and positioning the biopsy chamber proximate the cutting probe such that the biopsy chamber is configured to receive and store a biological sample.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a schematic view of an example vitrectomy system;

FIG. 2 is a schematic view of a biopsy chamber of the system shown in FIG. 1;

FIG. 3 is a cross-sectional view of the biopsy chamber of FIG. 2 and taken along line 3-3; and

FIG. 4 is a schematic view of the system shown in FIG. 1 inserted into an eye.

### DETAILED DESCRIPTION

This disclosure generally relates to systems for withdrawing and storing biopsy samples. For example, the system of one embodiment is an ophthalmic surgical system capable of harvesting undiluted vitreous from an eye. As further described below, a surgeon can draw a vitreous sample from the eye into a storage container or biopsy chamber and quickly remove the chamber from the system for vitreous analysis. In the described embodiment, the biopsy chamber is disposed in close proximity to a vitreous cutting probe. By locating the chamber close to the probe, less vitreous is removed from the eye than in conventional instruments. Embodiments of this disclosure may be used in various ophthalmic procedures such as pediatric ophthalmic procedures, and in particular, vitrectomy surgery. While the system described and shown in the drawings is adapted for ophthalmic procedures, systems consistent with this disclosure may also be adapted for and used in other surgical procedures.

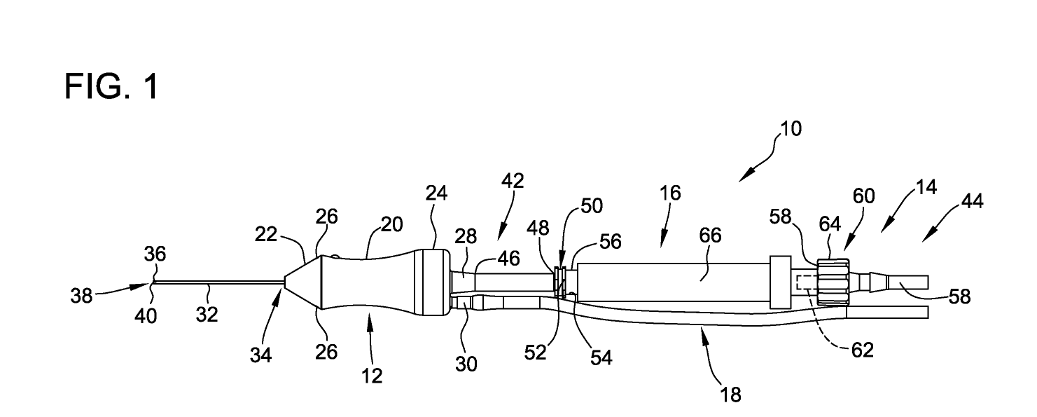

Referring to FIG. 1, a vitrectomy system of one embodiment is generally indicated by reference numeral 10. System 10 includes a cutting probe 12, a suction line 14, a biopsy chamber 16, and a fluid line 18. System 10 of this embodiment is configured for use in vitrectomy surgery, and more specifically for taking a vitreous biopsy sample from an eye of the human body. In the illustrated embodiment, the system enables a vitreous sample to be removed and stored without diluting the sample with irrigation fluid. Alternatively, the system 10 may be used for other surgical procedures. Additionally, as described below, system 10 can be selectively used to take a vitreous biopsy sample, or instead to perform a vitrectomy operation without collecting a biopsy sample.

In the example embodiment, cutting probe 12 includes a housing 20 having a nose 22, an end cap 24 and air vents 26. End cap 24 includes a suction port 28 coupled to suction line 14 and a fluid port 30 coupled to fluid line 18. Cutting probe 12 includes a probe needle 32 having a proximal end 34 coupled to housing 20 and a distal end 36 having a vitreous inlet 38. A cutting blade 40 is positioned within probe needle 30 to cut fibers of vitreous drawn within vitreous inlet 38.

Suction line 14 includes a first section 42 and a second section 44. First section 42 includes a first end 46 coupled to suction port 28 and a second end 48 having a female connector 50. In the exemplary embodiment, female connector 50 includes a body 52 having an inner diameter 54 and an outer diameter 56. Second section 44 includes a first end 58 having a male connector 60 and a second end coupled to a remote suction unit (not shown). Male connector 60 includes a Luer 62 and a threaded fitting 64. Male connector 60 is operable for connection with female connector 50 to create a fluid-tight seal therebetween, for example, when biopsy chamber 16 is removed from system 10 following a biopsy procedure. First and second sections 42, 44 of suction line 14 can be connected to one another when biopsy chamber 16 is removed from the system, thus enabling the biopsy chamber 16 to be removed from the system while the cutting probe 12 is still positioned within an incision in a body. As a result, a biopsy sample collected in biopsy chamber 16 can be removed from system 10 (e.g., to analyze the biopsy sample) without interrupting other surgical procedures for which system 10 is configured to perform (e.g., vitrectomies).

Fluid line 18 is coupled to fluid port 30 to provide a fluid (e.g., compressed air) to cutting probe 12. Pulsating pressurized fluid is delivered through fluid line 18 to reciprocate cutting blade 40 within probe needle 32 and is vented via air vents 26.

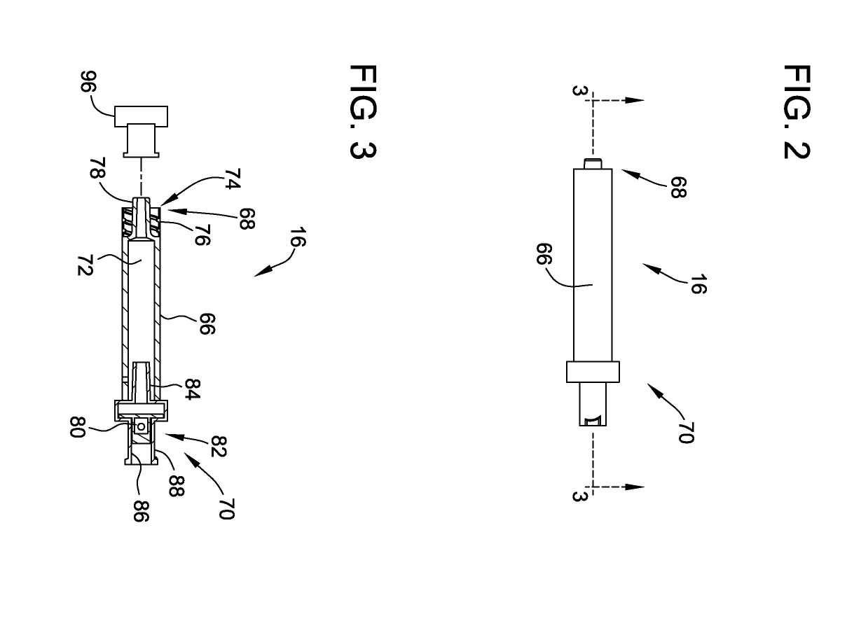

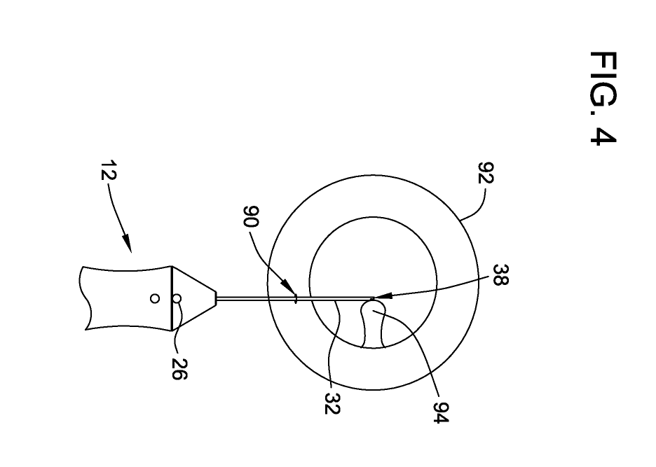

Referring to FIGS. 1-3, biopsy chamber 16 is coupled to suction line 14 between first section 42 and second section 44. Biopsy chamber includes a tubular body 66 having a first end 68 and a second end 70. Tubular body 66 includes a vitreous chamber 72 operable to receive and store an amount of vitreous from an eye of the human body. In the example embodiment, vitreous chamber 72 has a volume of approximately 1 mL. However, vitreous chamber 72 may have any volume that enables system 10 to function as described herein. Additionally, any number of biopsy chambers 16 may be coupled between first and second suction line sections 42 and 44 to draw and store a desired amount of vitreous.

Biopsy chamber first end 68 includes a male connector 74 having a threaded fitting 76 and a Luer entry port 78 fluidly connected to vitreous chamber 72. Male connector 74 is operable for connection with female connector 50 such that Luer 78 is received within inner diameter 54 of female connector 50 to create a fluid-tight seal therebetween. Biopsy chamber second end 70 includes a Luer activated valve 80 and a female connector 82 having an exit port 84 with an inner diameter 86 and an outer diameter 88. Female connector 82 is operable for connection with male connector 60 such that Luer 62 is received within inner diameter 86 of female connector 82 to create a fluid-tight seal therebetween. Luer 62 is operable to engage valve 80 to allow fluid to pass thereby.

In use, biopsy chamber 16 is fluidly coupled within suction line 14. In particular, biopsy chamber male connector 74 is coupled to female connector 50 of suction line first section 42, and first end 46 of suction line 14 is coupled to cutting probe suction port 28. Biopsy chamber female connector 82 is coupled to male connector 60 of suction line second section 44 such that Luer 62 of male connector 60 engages Luer activated valve 80 into an open position. In the example embodiment, the volume and/or length of suction line first section 42 is kept at a minimum to reduce the distance of travel to vitreous chamber 72. Biopsy chamber 16 is kept in close proximity to cutting probe 12 to reduce the volume of “wasted” vitreous between cutting probe 12 and chamber 16, which reduces the amount of vitreous removed from the eye. In the example embodiment, suction line first section 42 is between approximately 0.125 inches and approximately one inch, so that the biopsy chamber is within 0.125 inches and one inch of the cutting probe. In another embodiment, suction line first section 42 is approximately 0.75 inches, so that the chamber is within 0.75 inches of the cutting probe. Further, the volume enclosed by first section 42 of suction line 14 is less than about 0.5 mL, more suitably less than about 0.25 mL, and, more suitably about 0.1 mL. In alternative embodiments, the biopsy chamber 16 may be positioned at any suitable distance from cutting probe 12 that enables system to function as described herein. In one embodiment, for example, first section 42 of suction line 14 has a length of less than about 5 inches such that biopsy chamber 16 is positioned within about 5 inches of cutting probe 12.

By locating biopsy chamber 16 close to cutting probe 12 and limiting the volume of second section 42 of suction line 14, a sufficient amount of vitreous may be harvested without the need to provide irrigation fluid to the eye to prevent collapse. Without a supply of irrigation fluid to the eye, an undiluted vitreous sample may be drawn from the eye. Additionally, locating biopsy chamber 16 near cutting probe 12 enables system 10 to have an ergonomic design and facilitates handling of the cutting probe 12 during surgical procedures. Locating biopsy chamber 16 near cutting probe 12 also enables a surgeon to visually monitor the collection of biopsy samples without having to look away from the surgical site.

In an alternative embodiment, first section 42 of suction line 14 is omitted from system 10, and first end 68 of biopsy chamber 16 may be connected directly to cutting probe 12. More specifically, male connecter 74 is connected to suction port 28. Additionally or alternatively, male connecter 74 is connected to suction port 28 by an adapter (e.g., female connecter 50). Such embodiments facilitate locating biopsy chamber 16 near cutting probe 12, and reducing the volume of “wasted” vitreous between cutting probe 12 and chamber 16.

Suction line second section 44 is coupled to a remote suction unit operated by a surgeon, and fluid line 18 is coupled to probe fluid port 30 and to a remote pumping unit operated by the surgeon. The remote suction unit and the remote pumping unit may be a single unit.

In the example operation, as illustrated in FIG. 4, an incision 90 is made into an eye 92 and probe needle 32 is inserted therein into vitreous material 94. The surgeon operates the pumping/suction unit and suction line 14 draws vitreous 94 into vitreous inlet 38 while a fluid is delivered via fluid line 18 to operate reciprocating cutting blade 40 to cut the fibers of vitreous as they are drawn into probe needle 32. Vitreous is drawn through suction line first section 42 and into vitreous chamber 72. Once a desired amount of vitreous is drawn into biopsy chamber 16 (or multiple connected biopsy chambers), the pumping/suction unit is shut off and biopsy chamber male connector 74 is uncoupled from fluid line female connector 50. A cap 96 is coupled to male connector 74 to seal biopsy chamber male Luer entry port 78. Biopsy chamber female connector 82 is uncoupled from fluid line male connector 60, which closes Luer activated valve 80 to seal biopsy chamber exit port 84. Biopsy chamber 16 is thereby sealed, and removed from vitrectomy system 10 for analysis of the vitreous sample. Female connector 50 of fluid line first section 42 may be coupled to male connector 60 of fluid line second section 44 to provide suction to cutting probe 12 for further procedures, such as a vitrectomy.

The system 10 is configured such that the system can be selectively used to perform a biopsy (i.e., collect a sample of tissue), to perform a surgical procedure (e.g., a vitrectomy), or to perform both a biopsy and a surgical procedure. For example, biopsy chamber 16 may be removed from system 10, and first section 42 may be connected directly to second section 44 to perform a vitrectomy. Additionally, as described above, biopsy chamber 16 may be used to perform a biopsy operation and then be removed from system 10 in order to perform a vitrectomy without removing cutting probe 12 from an incision in a body.

In an alternative embodiment, system 10 includes a handle (not shown) to which cutting probe 12 and biopsy chamber 16 are secured. The handle assembly thus includes the cutting probe 12 and biopsy chamber 16 as an integral unit, thereby facilitating handling of system 10 during surgical procedures.

The handle may be fabricated from any suitable material (e.g., over-molded plastic) that enables system 10 to function as described herein. Biopsy chamber 16 is releasably secured to the handle such that biopsy chamber 16 can be removed (e.g., for analysis of a biopsied sample) without removing cutting probe 12 from an incision in a body. Suction line 14 and, more specifically, first and second sections 42, 44 of suction line 14, may also be secured to handle. In one embodiment, for example, suction line 14 is integrally formed in the handle to facilitate connecting and disconnecting biopsy chamber 16.

When biopsy chamber 16 is removed from the handle, an additional section of suction line (not shown) may be inserted between first and second sections 42, 44 so that, for example, additional surgical procedures or operations may be performed without biopsy chamber 16 connected to system 10. Alternatively, system 10 may include a switch valve (not shown) configured to divert suction from biopsy chamber 16 to a bypass section of suction line 14 such that cutting probe 12 is fluidly connected to suction unit when biopsy chamber 16 is removed from system 10. The switch valve may have any suitable configuration that enables the switch valve to selectively fluidly connect the suction unit to suction port 28 via one of the biopsy chamber 16 and the bypass section of suction line 14. In one embodiment, the switch valve is a three-port valve (e.g., a T-valve or a Y-valve), and is fluidly connected to suction port 28 of cutting probe 12, first end 68 of biopsy chamber 16, and the bypass section of suction line 14. The switch valve may be either manually or automatically actuated. In one embodiment, for example, the switch valve is automatically actuated upon connecting and/or disconnecting biopsy chamber 16 within system 10.

When introducing elements of the present invention or the embodiments thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the invention, it is intended that all matter contained in the above description and shown in the accompanying figures shall be interpreted as illustrative and not in a limiting sense.

What is claimed is:

1. A biopsy system comprising:
a cutting probe including a suction port;
a suction line connected to the suction port; and
a biopsy chamber fluidly connected to the cutting probe by the suction line, the biopsy chamber positioned proximate the cutting probe such that the biopsy chamber is configured to receive and store a biological sample free from irrigation fluid.

2. The system of claim 1, wherein the biopsy chamber is positioned sufficiently near the cutting probe such that the biopsy chamber is configured to receive a vitreous sample free from irrigation fluid.

3. The system of claim 1, wherein the biopsy chamber is positioned less than about 1.0 inches from the cutting probe.

4. The system of claim 1, wherein the biopsy chamber includes a tubular body having a first end and a second end, and the suction line includes a first section and a second section, the first section connected to the first end of the tubular body and the suction port, the second section connected to the second end of the tubular body and a suction unit.

5. The system of claim 4, wherein the first section of suction line has a length of less than about 1.0 inches.

6. The system of claim 4, wherein the second end of the tubular body includes an exit port comprising a Luer-actuated valve, the second section of suction line connected to the exit port.

7. The system of claim 6, wherein the second section of suction line includes a Luer configured to actuate the Luer-actuated valve to allow fluid to pass therethrough.

8. The system of claim 4, further comprising a cap adapted to be connected to the first end of the tubular body to seal the biological sample within the tubular body.

9. The system of claim 1, wherein the cutting probe further comprises a probe needle having a sample inlet and a cutting blade.

10. The system of claim 1, wherein the cutting probe further comprises at least one air vent.

11. The system of claim 1, wherein the cutting probe further comprises a fluid port, and wherein the system further comprises a fluid line connected to the fluid port.

12. A method of assembling a biopsy system including a cutting probe having a suction port, a suction line having a first section and a second section, and a biopsy chamber having a first end and a second end, the method comprising:
fluidly connecting the first end of the biopsy chamber to the suction port of the cutting probe with the first section of the suction line;
connecting the second end of the biopsy probe to the second section of the suction line; and
positioning the biopsy chamber proximate the cutting probe such that the biopsy chamber is configured to receive and store a biological sample.

13. The method of claim 12, wherein positioning the biopsy chamber includes positioning the biopsy chamber sufficiently near the cutting probe such that the biopsy chamber is configured to receive a vitreous sample free from irrigation fluid.

14. The method of claim 12, wherein positioning the biopsy chamber includes positioning the biopsy chamber less than about 1.0 inches from the cutting probe.

15. The method of claim 12, wherein the first section of suction line has a length of less than about 1.0 inches.

16. The method of claim 12, further comprising connecting the second section of suction line to a suction unit.

17. The method of claim 12, wherein the second end of the biopsy chamber includes an exit port including a Luer-actuated valve, and connecting the second end of the biopsy chamber includes connecting the second section of the suction line to the exit port.

18. The method of claim 12, further comprising connecting a fluid line to the cutting probe for reciprocating a cutting blade within the cutting probe.
